(19) United States
(12) Patent Application Publication
(10) Pub. No.: US 2011/0230750 A1
(43) Pub. Date: Sep. 22, 2011

(54) MEASURING APPARATUS

(75) Inventor: Jiro Tateyama, Yokohama-shi (JP)

(73) Assignee: CANON KABUSHIKI KAISHA, Tokyo (JP)

(21) Appl. No.: 13/048,666

(22) Filed: Mar. 15, 2011

(30) Foreign Application Priority Data
Mar. 19, 2010 (JP) 2010-063645

(51) Int. Cl. A61B 5/05

(52) U.S. Cl. 600/407

(57) ABSTRACT

The present invention provides a measuring apparatus having: a first probe including a first acoustic conversion element that transmits an ultrasound wave, receives the ultrasound wave after the ultrasound wave is reflected by an object, and converts the ultrasound wave into an analog signal; a second probe including a second acoustic conversion element that receives a photo-acoustic wave generated when light emitted from a light emitter is absorbed by the object and converts the photo-acoustic wave into an analog signal; a receiver that receives the analog signals converted by the first and second acoustic conversion elements and converts the analog signals into digital signals; and a switching unit that switches the acoustic conversion element from which the receiver receives the analog signal.

### BACKGROUND OF THE INVENTION

1. Field of the Invention

The present invention relates to a measuring apparatus for measuring biological information.

2. Description of the Related Art

Research into measuring apparatuses that use optical imaging techniques for obtaining information about an organism interior by causing light emitted onto the organism using a light source such as a laser to propagate through the organism interior is being pursued actively in medical fields. One of these optical imaging techniques is photo-acoustic imaging, otherwise known as PAT (Photo-acoustic Tomography). In this technique, an organism is irradiated with pulsed light emitted from a light source, and a photo-acoustic wave (typically an ultrasound wave) generated when energy from the pulsed light is absorbed by body tissue as the pulsed light propagates and diffuses through the organism interior is detected. Are suiting detection signal is then subjected to analysis processing to obtain an optical characteristic distribution, and in particular an optical energy absorption density distribution, of the organism interior.

A high-resolution optical characteristic value distribution is obtained with a photo-acoustic imaging apparatus, and therefore a photo-acoustic imaging apparatus is used to measure a substance concentration of the organism interior. A typical ultrasound wave measuring apparatus, on the other hand, is widely used to determine the existence of morphological features of the organism interior. Hence, by combining functional imaging for expressing the substance distribution of the body tissue and morphological imaging for expressing morphological features, the tissue can be characterized in more detail, while malignant tumors and the like can be diagnosed more accurately.

A measuring apparatus described in Japanese Patent Application Laid-Open No. 2005-021380 forms an image of organism information from an object by detecting not only an ultrasound echo but also a photo-acoustic wave generated on the basis of energy from light emitted into the object interior.

This conventional example will now be described using FIG. 3A. A light emitter 310 provided in the apparatus irradiates an organism with light. A probe 305 is a 1D probe on which 256 acoustic conversion elements for detecting photo-acoustic waves are disposed in one-dimensionally (1D). The acoustic conversion elements are piezoelectric elements made of PZT (lead zirconate titanate) or the like, for example, and are capable of receiving and detecting a photo-acoustic wave generated from a light absorbing body in the organism interior when the light absorbing body absorbs a part of the energy of the light emitted by the light emitter. The acoustic conversion elements also have a function for simultaneously outputting an ultrasound wave in response to control of a high voltage transmission pulser circuit (a 64CH transmitter 303). Hence, in this conventional example, the acoustic conversion elements are also used to transmit and receive ultrasound waves.

Further, when a linear scan is performed in the conventional example using the 1D probe, a linear scanning high voltage analog switch circuit (a high voltage SW circuit 314) that is operated by connecting only a required opening portion to a transmission/reception circuit is used. As shown in FIG. 4, this switch circuit performs a linear scan by connecting the 256 element 1D probe to a 64 channel transmission/reception circuit and performing ON/OFF switching operations to shift rightward one element at a time.

A weak analog signal (electric signal) detected by the acoustic conversion elements is then subjected to signal processing using a 64CH receiver 304. More specifically, the weak analog signal is amplified by a reception amplification circuit and then digitally sampled by an A/D converter. A resulting digital signal is transmitted to an image processor 311 for calculating optical characteristic value distribution information and so on relating to the organism.

An apparatus according to this conventional example uses the 1D probe in which the light emitter and the acoustic conversion elements are integrated, and therefore a photo-acoustic image and an ultrasound image of a substantially identical region to the tissue that receives the scan can be obtained simultaneously. In other words, a photo-acoustic image generated from the energy of the light emitted onto the object receiving the scan and an ultrasound image generated from an ultrasound wave emitted onto the object receiving the scan can be superimposed, and therefore a substance concentration distribution relative to morphological features of the tissue of the object can be learned.

In another conventional example described in Japanese Patent Application Laid-Open No. 2009-031268, acoustic conversion elements are disposed on a substrate in a two-dimensional (2D) array. More specifically, as shown in FIG. 3B, by employing a 2D probe 306 in which acoustic conversion elements are disposed in a two-dimensional array, a photo-acoustic wave corresponding to a three-dimensional region can be detected through a single light emission operation. As a result, a photo-acoustic image having a 3D structure can be generated through a single light emission operation.

### SUMMARY OF THE INVENTION

In the conventional example described in Japanese Patent Application Laid-Open No. 2005-021380, however, the integrated probe is used, and therefore the characteristics of the acoustic conversion elements cannot be separated into a photo-acoustic wave reception characteristic and an ultrasound wave transmission/reception characteristic. In the case of a conventional probe, for example, a 2D sector probe having a rough element pitch of approximately 1.0 to 2.0 mm is used to receive photo-acoustic waves so that an acoustic wave having a large surface area can be obtained at one time. To transmit and receive ultrasound waves, on the other hand, a 1D linear probe having a narrow element pitch of approximately 0.2 to 0.3 mm is used so that an acoustic wave of a small region can be obtained at a high resolution. Hence, a probe having optimum characteristics for both operations cannot be selected.

Further, with an integrated probe, a common circuit can be used to receive photo-acoustic waves and ultrasound waves. However, an ultrasound wave receiver is typically constituted by a high voltage driven pulser circuit, and therefore system noise generated when the pulser circuit is on standby may flow into the receiver. As a result, this inflowing system noise becomes problematic when detecting a photo-acoustic wave that is weaker than an ultrasound wave (an ultrasound echo) reflected by the object interior.

Furthermore, when a 2D probe is used to receive photo-acoustic waves, a transmission/reception circuit having an identical number of channels to the number of elements on the probe is required, and as a result, an increase in circuit scale occurs, making it difficult to suppress the cost and size of the apparatus. Moreover, in a case where the probes are separate but the transmitter is shared, inflowing system noise cannot be avoided.

The present invention has been designed in consideration of the circumstances described above, and an object thereof is to provide a technique for suppressing inflowing system noise in a measuring apparatus when a probe is switched to receive a photo-acoustic wave and an ultrasound wave.

This invention provides a measuring apparatus comprising:

a transmitter that determines a timing for transmitting an ultrasound wave;

a first probe including a first acoustic conversion element that transmits the ultrasound wave in response to an instruction from the transmitter, receives the ultrasound wave reflected in an object, and converts the ultrasound wave into an analog signal;

a light emitter;

a second probe including a second acoustic conversion element that receives a photo-acoustic wave generated when light emitted from the light emitter is absorbed by the object and converts the photo-acoustic wave into an analog signal;

a receiver that receives the analog signals converted by the first and second acoustic conversion elements and converts the analog signals into digital signals; and

a switching unit that performs a switch such that the receiver receives an analog signal from one of the first acoustic conversion element and the second acoustic conversion element and ensures that while the receiver is receiving an analog signal from one acoustic conversion element, the receiver does not receive an analog signal from another acoustic conversion element.

According to the present invention, inflowing system noise in a measuring apparatus can be suppressed when a probe is switched to receive a photo-acoustic wave and an ultrasound wave.

Further features of the present invention will become apparent from the following description of exemplary embodiments with reference to the attached drawings.

### BRIEF DESCRIPTION OF THE DRAWINGS

FIG. 1 is a block diagram showing the constitution of a measuring apparatus;

FIG. 2 is a view illustrating an operation of an apparatus according to a first embodiment;

FIG. 3 is a view illustrating reception data processing according to a conventional example;

FIG. 4 is a view showing a linear scan using a switch circuit;

FIG. 5 is a view illustrating an operation of an apparatus according to a second embodiment;

FIG. 6 is a timing chart of time sharing control; and

FIG. 7 is a view showing switching patterns of the time sharing control.

### DESCRIPTION OF THE EMBODIMENTS

The present invention is applied to a measuring apparatus that combines a measuring apparatus for receiving an ultrasound echo reflected by an object interior in response to a transmitted ultrasound wave and a measuring apparatus for receiving a photo-acoustic wave generated in the object interior when the object interior is irradiated with light. The received ultrasound wave or photo-acoustic wave can be used to form an image of the object interior. Further, in the present invention, an acoustic wave (elastic wave) generated in response to optical irradiation will be referred to as a “photo-acoustic wave”, while an acoustic wave transmitted from an acoustic conversion element and a reflected wave generated when the transmitted acoustic wave is reflected by the object interior will be referred to as an “ultrasound wave” or an “ultrasound echo”.

Embodiments will be described in detail below with reference to the drawings.

### First Embodiment

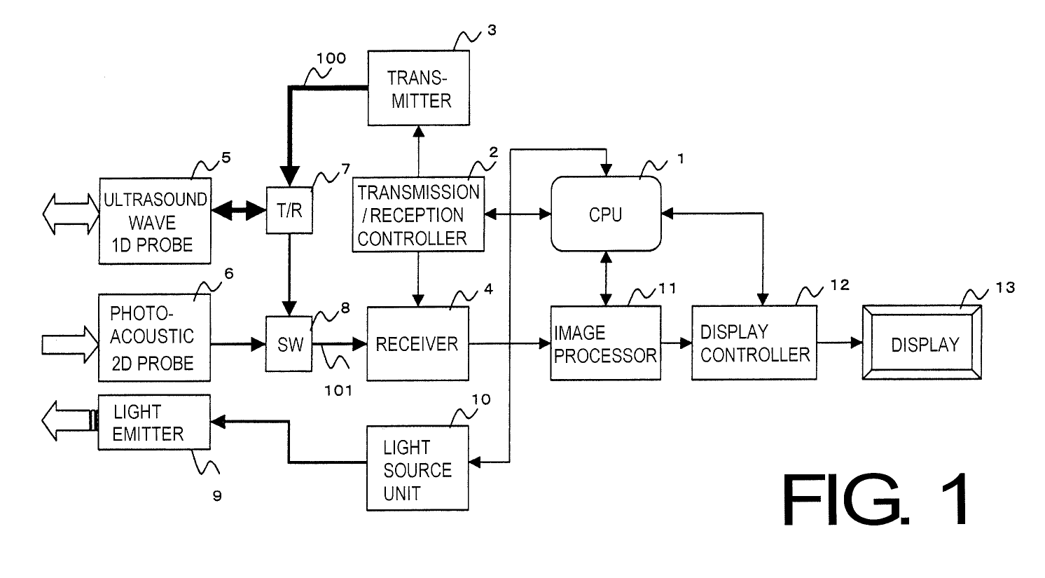

FIG. 1 is a block diagram of a measuring apparatus according to a first embodiment.

A CPU 1 performs main control of the measuring apparatus. A transmission/reception controller 2 performs beam forming control with regard to ultrasound wave transmission/reception. A transmitter 3 generates an ultrasound wave by issuing an instruction to drive a probe. A receiver 4 processes reception data detected by the probe. An ultrasound wave 1D probe (a first probe) 5 is configured to generate an ultrasound wave and detect an ultrasound echo, i.e. a reflected wave. A plurality of acoustic conversion elements (first acoustic conversion elements) provided on the ultrasound wave 1D probe are one-dimensionally arranged elements suitable for detecting ultrasound waves. A photo-acoustic 2D probe (a second probe) 6 is used only to detect a photo-acoustic signal. A plurality of acoustic conversion elements (second acoustic conversion elements) provided on the photo-acoustic 2D probe are two-dimensionally arranged elements suitable for detecting photo-acoustic waves. A bridge circuit (T/R) 7 applies a limiter to a high voltage signal output from the transmitter so that the high voltage signal converges with a detectable voltage value of the receiver. A switch circuit (SW) 8 switches between the ultrasound wave 1D probe 5 and the photo-acoustic 2D probe 6. A light emitter 9 irradiates an organism with light. A light source unit 10 drive-controls the light emitter. An image processor 11 calculates concentration information from the photo-acoustic wave and morphology information from the ultrasound echo and generates image data. A display controller 12 performs a scan conversion. A display 13 displays an image.

A basic imaging operation using an ultrasound wave will now be described. When a user begins an operation by bringing the probe into contact with an object (an organism), the transmitter 3 determines an ultrasound wave transmission timing and issues an instruction to drive the ultrasound wave 1D probe 5. The probe generates an ultrasound wave in the organism. The ultrasound wave advances through the organism quickly until it impinges on a hard object, whereby an ultrasound echo is reflected. The probe detects the ultrasound echo, calculates a distance from a time interval between transmission of the ultrasound wave and reflection of the ultrasound echo, and visualizes the interior of the organism. In other words, a morphological image expressing a substance distribution of body tissue can be formed.

A basic imaging operation using a photo-acoustic wave will now be described. First, the light emitter 9 irradiates the organism with pulsed light. Next, the photo-acoustic 2D probe 6 detects an acoustic wave generated when the body tissue absorbs energy from the pulsed light that propagates and diffuses through the organism interior. A resulting detection signal is then subjected to analysis processing by the image processor 11, whereby an optical characteristic distribution, and in particular an optical energy absorption density distribution, of the organism interior can be obtained. The image processor then generates image data on the basis of corresponding data. In other words, a functional image expressing the substance distribution of the body tissue can be formed.

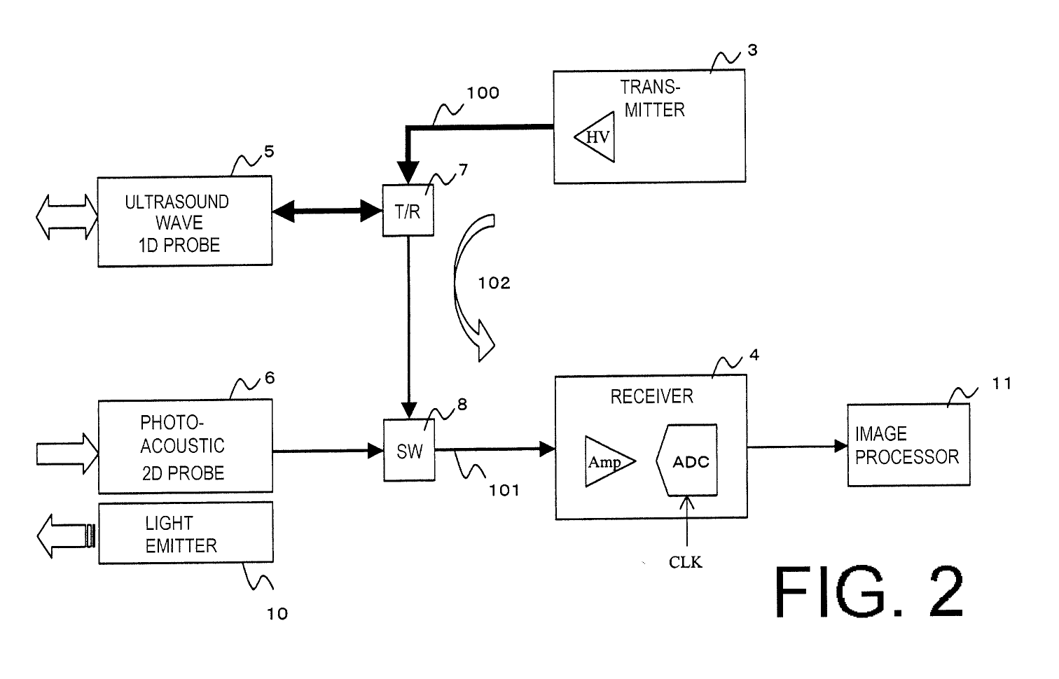

FIG. 2 is a view illustrating an operation of the apparatus according to the first embodiment. The transmitter 3 is a high voltage driven pulser circuit constituted by an HV-CMOS. The transmitter 3 generates an ultrasound wave by determining a timing for driving the ultrasound wave 1D probe on the basis of a pulse and issuing an instruction thereto. The receiver 4 generates a digital signal by amplifying an ultrasound echo or a weak signal of a photo-acoustic wave detected by the probe using a pre-amp circuit (Amp) and performing digital sampling thereon in synchronization with a clock CLK using an A/D converter circuit (ADC).

A high voltage signal 100 output to the probe from the transmitter exceeds an allowable voltage value of a detection signal 101 input into the receiver, and therefore a limiter must be applied thereto. Hence, in this embodiment, the high voltage signal 100 is converged to a voltage value within ±5 V using the diode bridge circuit (T/R). Since the transmitter is constituted by a high voltage driven circuit, a holding current must be applied continuously at this time to keep the CMOS circuit, which operates at a ±100 V level, in an operable condition at all times. As a result, system noise 102, albeit very small, is generated during operation holding.

In this embodiment in particular, the receiver 4 is used to detect signals corresponding to both the ultrasound wave and the photo-acoustic wave, and therefore the system noise 102 generated by the transmitter may become mixed into the detection signal 101 from the photo-acoustic probe during photo-acoustic wave detection. Since the photo-acoustic wave is weaker than the ultrasound echo, system noise intermixing has a greater effect.

Hence, in this embodiment, the switch circuit (SW) 8 that performs a switch by selecting one of the ultrasound wave probe 5 and the photo-acoustic probe 6 is provided on an input end of the receiver. The switch circuit 8 switches a detection source for the detection signal 101 input into the receiver between a period for detecting the photo-acoustic wave and a period for detecting the ultrasound echo of the ultrasound wave. More specifically, during the period for detecting the photo-acoustic wave, only the signal detected by the photo-acoustic 2D probe 6 is set as the detection signal and the signal detected by the ultrasound wave 1D probe 5 is not input. As a result, while one of the analog signals (electric signals) originating from the ultrasound wave and the photo-acoustic wave is being received by the receiver, the other is not received.

By providing the switch circuit to separate the detection signals of the photo-acoustic wave and the ultrasound echo in this manner, the signal from the ultrasound wave transmitter is prevented from flowing into the receiver during the period for detecting the photo-acoustic wave. As a result, the system noise generated during the operation of the transmitter can be suppressed, leading to an improvement in measurement precision.

### Second Embodiment

With the apparatus constitution of the first embodiment, the number of elements on the probe must be identical to the number of circuits in the transmitter/receiver.

However, in recent apparatuses that employ a 1D linear probe and a 2D array probe having a large number of elements, the number of probe elements has increased greatly, and therefore increases in the size and cost of the apparatus have become problematic. Accordingly, it has become necessary to take measures to reduce the number of circuits in the transmitter/receiver.

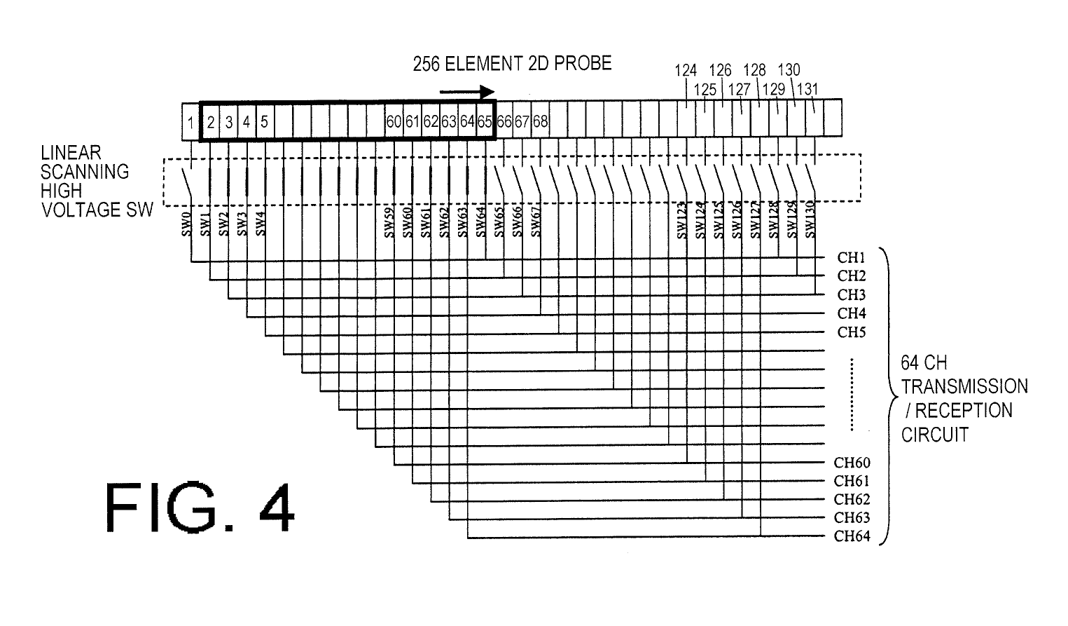

In a 1D linear probe, as described above with reference to FIG. 4, the number of circuits can be reduced to ¼, from 256 channels to 64 channels, for example, by employing a linear scanning high voltage switch circuit. However, a similar method cannot be employed for a 2D array probe.

Hence, in this embodiment, a measuring apparatus constituted to be capable of both suppressing system noise and reducing circuit scale will be described.

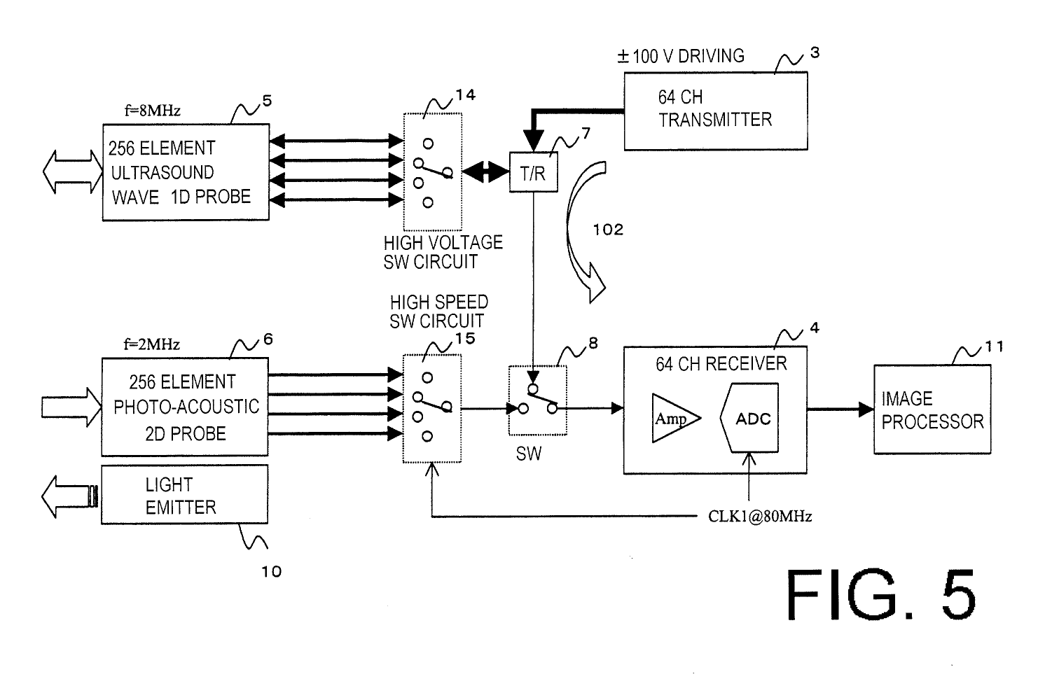

FIG. 5 is a view illustrating a measuring apparatus according to a second embodiment. The constitution of this apparatus will be described below, focusing on differences with the apparatus according to the first embodiment shown in FIG. 2. The transmitter 3 and the receiver 4 are constituted to perform 64 CH transmission/reception. 256 acoustic conversion elements are arranged one-dimensionally on the ultrasound wave 1D probe 5. 256 acoustic conversion elements are arranged two-dimensionally on the photo-acoustic 2D probe 6. A high voltage switch circuit (high voltage SW circuit) 14 is disposed between the bridge circuit (T/R) 7 and the ultrasound wave 1D probe 5. A high speed switch circuit (high speed SW circuit) 15 is disposed between the switch circuit (SW) 8 and the photo-acoustic 2D probe 6.

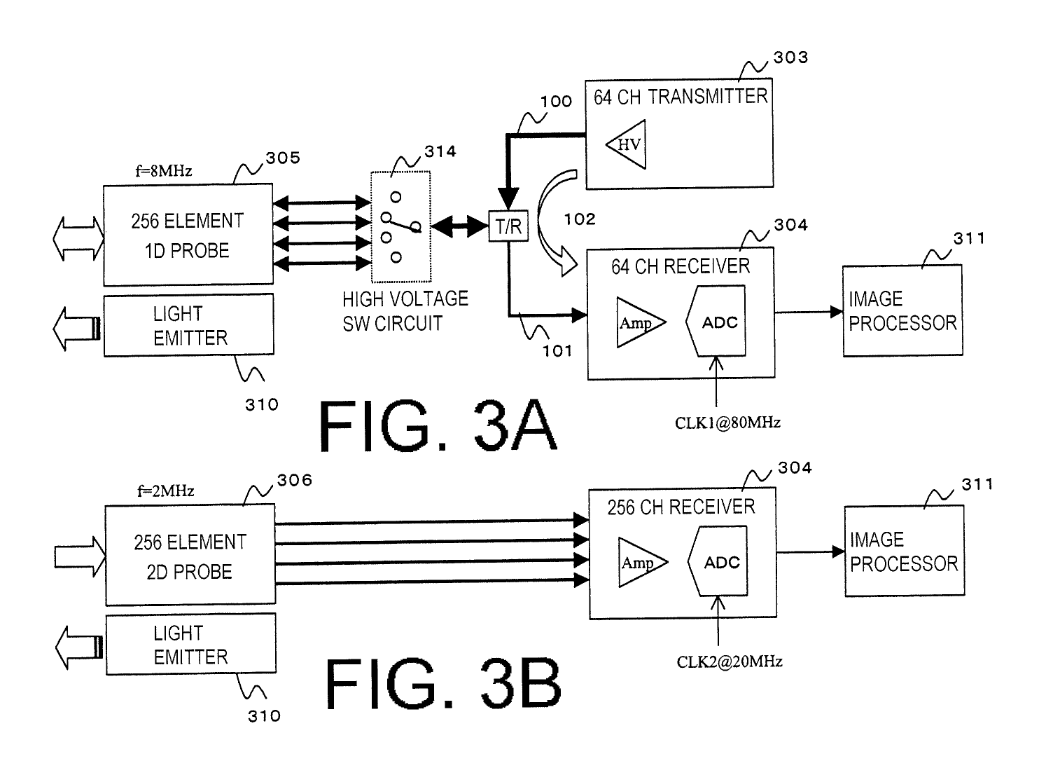

When the characteristics of the two probes are compared, as described above with reference to FIG. 3, the 1D linear probe for transmitting and receiving ultrasound waves has a narrow element pitch of approximately 0.25 mm in order to obtain an ultrasound wave of a small region at a high resolution. Accordingly, a center frequency of 8 MHz is set as the frequency characteristic of the probe. The 2D array probe for receiving photo-acoustic waves, on the other hand, has a rough element pitch of approximately 1.0 mm so that an acoustic wave having a large surface area can be obtained at one time, and a center frequency of 2 MHz is set as the frequency characteristic of the probe.

An optimum sampling frequency of the receiver is said to be approximately 8 to 10 times the center frequency of the probe. Here, as ten times the center frequency, an 80 MHz sampling clock is input as a CLK 1 in the 1D probe and a 20 MHz sampling clock is input as a CLK 2 in the 2D probe.

When a photo-acoustic signal is received according to the second embodiment, the high speed switch circuit 15 operates at an 80 MHz clock, which is four times greater than the 20 MHz clock of the photo-acoustic 2D probe 6. The A/D converter of the receiver 4 is also operated at 80 MHz. As a result, signals of photo-acoustic waves corresponding to four elements can be detected at a 20 MHz timing of the photo-acoustic 2D probe 6. Further, when the ultrasound wave 1D probe 5 is used to transmit and receive the ultrasound echo, the number of circuits can be reduced by providing the linear scanning high voltage switch circuit 14, as described with reference to FIG. 4.

Note that since the operating voltage of the signal output from the transmitter is high, a switch device that is only capable of raising an operating frequency to several MHz, which is a typical specification of a high voltage switch, is used as the high voltage switch circuit 14. On the other hand, a limit is applied to the operating voltage by the bridge circuit 7, and therefore a switch device capable of a high speed operation at up to several GHz may be selected as the high speed switch circuit 15. Further, the number of probe elements, the number of channels used during transmission and reception, and the operating clocks are not limited to those described in this embodiment and may be selected according to necessity.

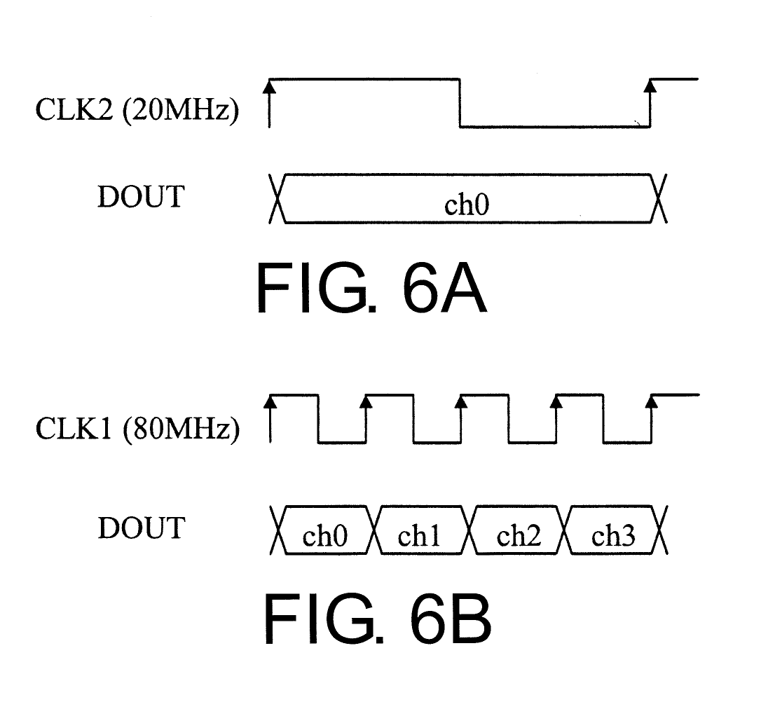

FIG. 6 is a timing chart showing time sharing control. FIG. 6A shows an example in which sampling is performed at 20 MHz without using the high speed switch circuit and output data correspond to a single element. FIG. 6B, on the other hand, shows an example in which sampling is performed at 80 MHz using the high speed switch circuit and the output data correspond to four elements. In other words, time sharing control is performed by driving both a switch clock of the high speed switch circuit 15 and a sampling clock of the A/D converter circuit of the receiver 4 at 80 MHz, i.e. at the same clock CLK 1. As a result, the number of circuits in the receiver can be reduced to ¼, from 256 channels to 64 channels.

A switch connection pattern for the 2D array probe employed at this time will now be described. As shown in FIG. 6, the detection signals of the four elements obtained in the time sharing control are sampled at respectively shifted phases, and therefore interpolation processing must be performed to align the phases before the signals are transmitted to the image processor 11. Linear interpolation or the like may be used as the interpolation processing, but when the number of probe elements is large, the interpolation processing must be simplified in order to reduce the processing time.

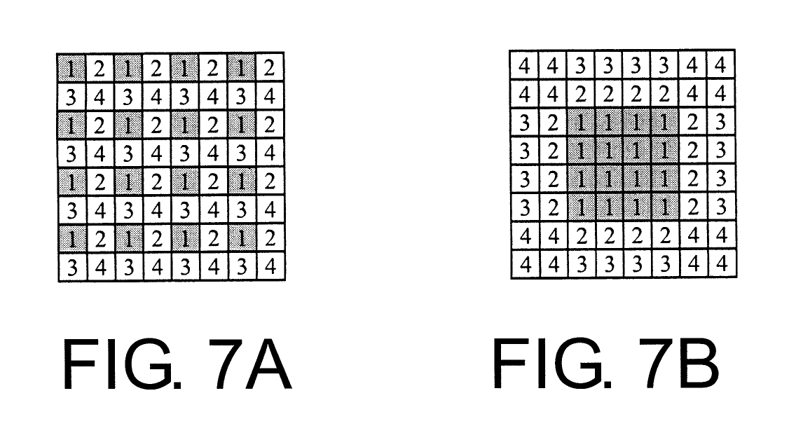

FIG. 7 is a view showing switch patterns of the time sharing control. FIG. 7A is a view of a staggered pattern in which switch circuits formed from sets of four elements are constituted by adjacent groups, and FIG. 7B is a pattern view of a radial layout.

In FIG. 7A, the four-element switch circuits are divided according to adjacent groups, and therefore the interpolation processing can be simplified by performing calculation processing using a common interpolation formula for each set of four elements. FIG. 7B shows a radial layout, and therefore the interpolation processing can be simplified by dividing the circuits into group units of a first element, a second element, a third element, and a fourth element, and performing calculation processing using a common interpolation formula for each group.

As described above, in the measuring apparatus according to this embodiment, the number of channels can be reduced and the circuit scale can be suppressed by providing the linear scanning high voltage switch circuit 14 and the time sharing control high speed switch circuit 15. As a result, reductions in the scale and cost of the apparatus can be achieved. Further, at this time, the switch circuit 8 operates in a similar manner to the first embodiment. Accordingly, while one of the analog signals originating from the ultrasound wave and the photo-acoustic wave is being received by the receiver, the other is not received. In other words, the transmitter 3 and the receiver 4 are not connected during reception of the photo-acoustic wave, and therefore system noise can be prevented from flowing in from the transmitter.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-063645, filed on Mar. 19, 2010, which is hereby incorporated by reference herein in its entirety.

What is claimed is:

1. A measuring apparatus comprising:
a transmitter that determines a timing for transmitting an ultrasound wave;
a first probe including a first acoustic conversion element that transmits the ultrasound wave in response to an instruction from the transmitter, receives the ultrasound wave reflected in an object, and converts the ultrasound wave into an analog signal;
a light emitter;
a second probe including a second acoustic conversion element that receives a photo-acoustic wave generated when light emitted from the light emitter is absorbed by the object and converts the photo-acoustic wave into an analog signal;
a receiver that receives the analog signals converted by the first and second acoustic conversion elements and converts the analog signals into digital signals; and
a switching unit that performs a switch such that the receiver receives an analog signal from one of the first acoustic conversion element and the second acoustic conversion element and ensures that while the receiver is receiving an analog signal from one acoustic conversion element, the receiver does not receive an analog signal from another acoustic conversion element.

2. The measuring apparatus according to claim 1, wherein the first probe is formed such that a plurality of the first acoustic conversion elements are arranged one-dimensionally and the second probe is formed such that a plurality of the second acoustic conversion elements are arranged two-dimensionally.

3. The measuring apparatus according to claim 2, wherein a number of channels on which the receiver can receive the analog signals is smaller than a number of the first acoustic conversion elements, and
the measuring apparatus further comprises a switch circuit that connects the first acoustic conversion element to the channel of the receiver to enable reception of an analog signal on the channel and switches the first acoustic conversion element connected to the channel of the receiver in sequence.

4. The measuring apparatus according to claim 2, wherein a number of channels on which the receiver can receive the analog signals is smaller than a number of the second acoustic conversion elements, and
the measuring apparatus further comprises a switch circuit that connects the second acoustic conversion element to the channel of the receiver to enable reception of an analog signal on the channel and switches the second acoustic conversion element for transmitting a signal to the channel of the receiver through time sharing.
